SEQUENCE LISTING (10 sequences)

SEQ ID NO: 1 - DNA, 20 bp, Artificial Sequence
  antisense oligonucleotide
actcctccagcacctcgttg

SEQ ID NO: 2 - DNA, 20 bp, Artificial Sequence
  antisense oligonucleotide
gcttgccaggtcgtaggtca

SEQ ID NO: 3 - DNA, 19 bp, Artificial Sequence
  antisense oligonucleotide
actcctccagcacctcgtt

SEQ ID NO: 4 - DNA, 20 bp, Artificial Sequence
  antisense oligonucleotide
cttcatctccttgttgccct

SEQ ID NO: 5 - DNA, 20 bp, Artificial Sequence
  antisense oligonucleotide
gtactcctccagcacctcgt

SEQ ID NO: 6 - DNA, 3459 bp, Homo sapiens
ggccggcggcggcggcggcggctccgctccgcactgcccggcgccgcctcgccatggacgcgcgcgggggcggcgggcggcccggggagagcccgggcgcgacccccgcgccggggccgccgccgccgccgccgcccgcgcccccccaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttatcgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtgaccctcgccgaccgccccgcgggcccaggcgggccgggggcggggccgtcatccagaccaaagccatgccattgcgctgccccggccgccagtccgcccagaagccatagacgagacgtaggtagccgtagttggacggacgggcagggccggcggggcagccccctccgcgcccccggccgtcccccctcatcgccccgcgcccacccccatcgcccctgcccccggcggcggcctcgcgtgcgagggggctcccttcacctcggtgcctcagttcccccagctgtaagacagggacggggcggcccagtggctgagaggagccggctgtggagccccgcccgccccccaccctctaggtggcccccgtccgaggaggatcgttttctaagtgcaatacttggcccgccggcttcccgctgcccccatcgcgctcacgcaataaccggcccggcccccgtccgcgcgcgtcccccggtgacctcggggagcagcaccccgcctccctccagcactggcaccgagaggcaggcctggctgcgcagggcgcgggggggaggctggggtcccgccgccgtgatgaatgtactgacgagccgaggcagcagtgcccccaccgtggccccccacgccccattaacccccacacccccattccgcgcaataaacgacagcattggcgccaaaaaaaaaaaaaaaaaaaaaaaaaaaaaaaaaaaaaaaaaaaaaaaaaaaaa

SEQ ID NO: 7 - DNA, 21 bp, Artificial Sequence
  Primer
cgcctgatccgctacatccat

SEQ ID NO: 8 - DNA, 23 bp, Artificial Sequence
  Primer
agtgcgaaggagtacagttcact

SEQ ID NO: 9 - protein, 204 aa, Homo sapiens
SAGDVDTLAFDGRTFVEYLNAVTESELANEIPVPETLDSGALHEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 10 - protein, 2067 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESELANEIPVPETLDSGALHEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

(19) United States
(12) Patent Application Publication
(10) Pub. No.: US 2016/0274126 A1
(43) Pub. Date: Sep. 22, 2016

(54) BIOMARKERS FOR THE DIAGNOSIS OF INTERSTITIAL CYSTITIS

(71) Applicant: WILLIAM BEAUMONT HOSPITAL, Royal Oak, MI (US)

(72) Inventors: MICHAEL B. CHANCELLOR, PITTSBURG, PA (US); PRADEEP TYAGI, PITTSBURG, PA (US); VIKAS TYAGI, NOIDA (IN)

(21) Appl. No.: 14/442,276

(22) PCT Filed: Nov. 11, 2013
(86) PCT No.: PCT/US13/69440

§ 371 Date: May 12, 2015

Related U.S. Application Data
(60) Provisional application No. 61/725,599, filed on Nov. 13, 2012.

(51) Int. Cl. G01N 33/68; C12N 15/113; C12Q 1/68

(52) U.S. Cl. CPC G01N 33/6893; C12Q 1/6883; G01N 33/6872; C12N 15/1138; C12Q 2600/158; C12Q 2600/106; C12Q 2600/112; G01N 2333/705; G01N 2333/4722; G01N 2800/34; C12N 2310/11; C12N 2320/30

(57) ABSTRACT

Disclosed are methods for the detection or diagnosis of a patient having symptoms of interstitial cystitis or overactive bladder. Also disclosed are HCN2 modulators and methods for the treatment of interstitial cystitis or overactive bladder.

### CROSS REFERENCE TO RELATED APPLICATION

This application claims priority to U.S. Provisional Application Ser. No. 61/725,599 filed Nov. 13, 2012. The entire content of U.S. Provisional Application Ser. No. 61/725,599 is incorporated herein by reference.

### INCORPORATION BY REFERENCE OF MATERIAL SUBMITTED ELECTRONICALLY

Incorporated by reference in its entirety is a computer-readable sequence listing submitted concurrently herewith and identified as follows: One 26 KB ASCII (Text) file named “5475-346886 Seq_Listing_ST25.txt,” created on Nov. 11, 2013, at 12:48 pm.

### BACKGROUND

Disorders of the urinary bladder may be associated with conditions such as disease, aging or infection but often are idiopathic. There are a number of urological diagnoses, including overactive bladder (OAB), interstitial cystitis (IC) and underactive bladder (UAB) that have overlapping lower urinary tract symptoms (LUTS). These symptoms include pelvic pain, pressure in the bladder, discomfort in the bladder or in the pelvic region, frequent urination, nocturia, decreased force of urination, urgency, urinary incontinence, pain during sexual intercourse.

Urge incontinence is seen mostly in OAB while bladder and pelvic pain is seen mostly in IC. Hypersensitive bladder is also used by some doctors when referring to OAB or IC as there is no one symptom that equates with a specific diagnosis, but rather a spectrum of diseases where the sensory and/or motor component of the bladder function is increased. Some symptoms more commonly associated with UAB include hesitancy, sensation of incomplete emptying, straining to void and recurrent infections. UAB can also occur with sensory neuropathy as seen in diabetic bladder dysfunction. Hyposensitivity is used sometime to refer to UAB, detrusor underactivity, paralysis of bladder, atonic bladder, floppy or lazy bladder. Causes of OAB and UAB may include neurogenic, myogenic, aging and medication side effects. Bladder smooth muscle is innervated by efferent motor nerve and sensory nerves

It is estimated that between three and eight million women in America are affected by interstitial cystitis (IC). Although the disease is most common in women, it also affects one to four million American men and can occur in children, although few statistics on pediatric cases exist. Interstitial cystitis is characterized by recurring and often significant pelvic pain, pressure and discomfort in the bladder and pelvic region; frequent urination; and increased urinary urgency.

For patients with severe interstitial cystitis, the disease can cause unrelenting pain and the need to urinate up to 60 times a day and throughout the night. These symptoms can prevent people from leaving the house or even riding in a car, making it impossible for them to work. According to the Association of Reproductive Health Professionals, over time the impact of interstitial cystitis can affect a patient's sleep, career, family life and sexual relationships, often leading to depression and a severely reduced quality of life.

The societal toll of the disease is significant as well. According to the most recent data available from the National Institute of Diabetes and Digestive and Kidney Diseases, interstitial cystitis (and the related painful bladder syndrome) was responsible for more than four million outpatient physician or clinic visits in 2000 and an outlay of $65.9 million, excluding missed work and lost productivity. There is little doubt those numbers would be significantly higher 12 years later.

IC, OAB and UAB may be associated with sensory dysfunction and/or motor dysfunction, however, because there is no reliable test for diagnosis, consistently effective treatments also are lacking, meaning sufferers may go through multiple approaches to care, usually on a trial and error basis, and often without finding any relief at all.

Thus, there is a significant need for reliable tests to aid the physician in selecting appropriate treatments patients having interstitial cystitis, overactive bladder or underactive bladder conditions.

### SUMMARY

The present disclosure provides methods for diagnosis of urinary disorders associated with an increased gene expression of HCN2 and/or increased levels of c-terminal agrin fragment (CAF). In particular, the present disclosure provides methods for diagnosis of overactive bladder, interstitial cystitis, or recurrence of interstitial cystitis, in a patient having symptoms of a urinary disorder. Also provided are methods for the detection and diagnosis of underactive bladder in a patient having symptoms of a urinary disorder. Methods are provided that enable a physician or other healthcare professional to select the appropriate treatment for a patient having symptoms of a urinary disorder.

The disclosure further provides for methods for the diagnosis of a disorder in a patient having one or more symptoms of interstitial cystitis comprising: (a) obtaining a biological sample from the patient, (b) measuring the level of an HCN2 gene expression product in the biological sample to obtain an HCN2 biomarker measurement and (c) using the HCN2 biomarker measurement to diagnose the disorder, wherein if the HCN2 biomarker measurement indicates an increase in the level of the HCN2 gene expression product, the patient is diagnosed as having interstitial cystitis.

The disclosure also provides for methods for the diagnosis of a disorder in a patient having one or more symptoms of overactive bladder comprising: (a) obtaining a biological sample from the patient, (b) measuring the level of an HCN2 gene expression product in the biological sample to obtain an HCN2 biomarker measurement and (c) using the HCN2 biomarker measurement to diagnose the disorder, wherein if the HCN2 biomarker measurement indicates an increase in the level of HCN2 gene expression product, the patient is diagnosed as having as having overactive bladder.

Also provided are methods for the diagnosis of a disorder in a patient one or more symptom of interstitial cystitis, overactive bladder, or underactive bladder comprising (a) obtaining a biological sample from the patient, (b) measuring the level of an HCN2 gene expression product in the biological sample to obtain an HCN2 biomarker measurement and (c) using the HCN2 biomarker measurement to diagnose the disorder, wherein if the HCN2 biomarker measurement indicates an increase in the level of HCN2 gene expression product, the patient is diagnosed as having as having interstitial cystitis, overactive bladder, or underactive bladder.

Also described are methods for diagnosing a patient having one or more symptom of interstitial cystitis, overactive bladder, or underactive bladder comprising (a) obtaining a biological sample from the patient, (b) obtaining a measurement of the level of c-terminal agrin fragment (CAF) in the biological sample to provide a CAF biomarker measurement, (c) using the CAF biomarker measurement to diagnose the patient as having interstitial cystitis, overactive bladder, or underactive bladder.

Further described are methods for diagnosing a patient having one or more symptom of interstitial cystitis, overactive bladder, or underactive bladder comprising (a) obtaining a biological sample from the patient, (b) obtaining a measurement of the level of c-terminal agrin fragment (CAF) in the biological sample to provide a CAF biomarker measurement, (c) obtaining a measurement of the level of a HCN2 gene expression product in a biological sample, (d) comparing the level of a HCN2 gene expression product to an HCN2 control level to provide an HCN2 biomarker measurement, and (e) using both the CAF biomarker measurement and the HCN2 biomarker measurement to diagnose the patient as having interstitial cystitis, overactive bladder, or underactive bladder.

Also described in the present disclosure are methods for a selecting a treatment regimen for a patient having one or more symptoms of interstitial cystitis or overactive bladder comprising: (a) obtaining a biological sample from the patient, (b) measuring the level of an HCN2 gene expression product in the biological sample to obtain an HCN2 biomarker measurement and (c) using the HCN2 biomarker measurement to select a treatment regimen for the patient.

Further provided are methods for selecting a treatment regimen for a patient having one or more symptom of interstitial cystitis, overactive bladder, or underactive bladder comprising (a) obtaining a biological samples from the patient, (b) obtaining a measurement of the level of CAF in the biological sample to provide a CAF biomarker measurement, (c) using the CAF biomarker measurement to select a treatment regimen for the patient.

Also provided are methods for selecting a treatment regimen for a patient having one or more symptom of interstitial cystitis, overactive bladder, or underactive bladder comprising (a) obtaining a biological samples from the patient, (b) obtaining a measurement of the level of CAF in the biological sample to provide a CAF biomarker measurement, (c) obtaining a measurement of the level of a HCN2 gene expression product in a biological sample, (d) comparing the level of a HCN2 gene expression product to an HCN2 control level to provide an HCN2 biomarker measurement, and (e) using both the CAF biomarker measurement and the HCN2 biomarker measurement to select a treatment regimen for the patient.

### BRIEF DESCRIPTION OF THE DRAWINGS

FIG. 1 is a graph showing the urine concentration of c-terminal agrin fragment (pM) in OAB patients relative to asymptomatic controls.

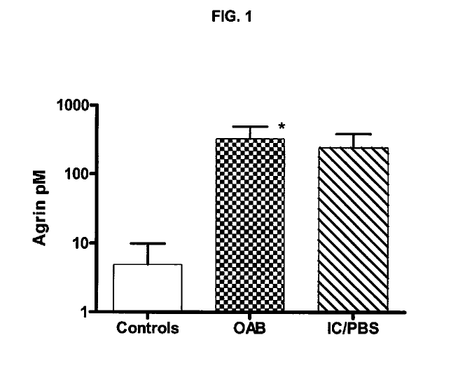

### DETAILED DESCRIPTION

### Definitions

Unless defined otherwise, all technical and scientific terms used herein have the meaning commonly understood by one of ordinary skill in the art to which this invention belongs.

HCN2 is an abbreviation for hyperpolarization activated cyclic nucleotide-gated potassium channel 2.

“HCN2 protein” means a gene expression product of the HCN2 gene or a functional fragment thereof that comprises amino acids. “HCN2 protein” and “HCN2 channel protein” are used interchangeably herein.

“HCN2 mRNA” means mRNA transcribed from the HCN2 gene.

“HCN2 gene expression product” means an HCN2 protein or an HCN2 mRNA, as defined herein.

“HCN2 modulator” means a molecule or composition, alone or in combination with one or more other molecules or compositions, or a therapy, which modulates the expression of an HCN2 gene expression product. An HCN2 modulator either increases or decreases the expression of an HCN2 gene expression product

Interstitial cystitis (IC) is also referred to as Interstitial cystitis/painful bladder syndrome (IC/PBS) and these terms are used interchangeably herein.

“Biological sample” is any fluid or other material derived from the body of a normal or diseased subject, such as urine, blood, or tissue, for example a biopsy sample.

The nucleotide sequence of human HCN2 has the GenBank accession number NM_01194.3. The sequence is provided as SEQ ID N0:6.

Agrin is cleaved by neurotrypsin. Cleavage of agrin at the 8-site releases C-terminal Agrin fragment (CAF, approx 22 kDa). As used herein the “c-terminal agrin fragment” or “CAF” refers to the 22 kDa c-terminal agrin portion of the human agrin protein corresponding to amino acids 1864-2067 (SEQ ID No: 9) of the human agrin protein, of the amino acid sequence as provided by GenBank accession number 000468.5 (SEQ ID No: 10) or a detectable fragment thereof.

“cDNA” refers to DNA oligonucleotide sequences that are completely or partially complementary; or completely or partially/substantially identical to RNA sequences. cDNA sequences identical to the mRNA sequence are often produced using a cDNA oligonucleotide with a complementary sequence as a template, for instance, using PCR, or any other synthetic method known in the art.

As used herein, the term “increase” or “increased” refers to a statistically significant and measurable increase in the biomarker level compared to a control level. The increase is preferably an increase of at least about 10%, or an increase of at least about 20%, or an increase of at least about 30%, or an increase of at least about 40%, or an increase of at least about 50%.

As used herein, the term “higher” in reference to a biomarker measurement refers to a statistically significant and measurable difference in the level of a biomarker measurement compared with a control level where the biomarker measurement is greater than the control level. The difference is preferably at least about 10%, or at least about 20%, or of at least about 30%, or of at least about 40%, or at least about 50%.

As used herein, the term “reduce” or “reduces” refers to a statistically significant and measurable reduction in the biomarker level compared to a control level. The reduction is preferably a reduction of at least about 10%, or a reduction of at least about 20%, or a reduction of at least about 30%, or a reduction of at least about 40%, or a reduction of at least about 50%.

As used herein, the term “lower” in reference to a biomarker measurement refers to a statistically significant and measurable difference in the level of a biomarker measurement compared with a control level where the biomarker measurement is less than the control level. The difference is preferably at least about 10%, or at least about 20%, or of at least about 30%, or of at least about 40%, or at least about 50%.

As used herein, the term “normalize” or “normalizes” refers to a modulation of the level of a biomarker product to within the normal range for levels of the biomarker product in a control subject.

The phrase “an effective amount” means an amount of an agent of the present invention, or a pharmaceutically acceptable salt thereof that is sufficient to inhibit, halt, or allow an improvement in the condition being treated when administered alone or in conjunction with another pharmaceutical agent. For example in a human an effective amount can be determined experimentally in a clinical setting, for the particular disease and subject being treated. It should be appreciated that determination of proper dosage forms, dosage amounts and routes of administration is within the level of ordinary skill in the pharmaceutical and medical arts.

The terms “treatment”, “treating”, “treat” and the like, refer to obtaining a desired pharmacologic and/or physiologic effect such as mitigating a symptom of a disorder or a disorder in a host and/or reducing, inhibiting, or eliminating a particular characteristic or event associated with a disorder (e.g., interstitial cystitis). Thus, the term “treatment” includes, preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted.

“Primer” means an oligonucleotide sequence that is capable of initiating or facilitating transcription or translation of a template oligonucleotide by binding or hybridizing to a template or target oligonucleotide. In some instances, a primer may contain one or more sequences that are complementary to the template or target oligonucleotide.

“Control level,” “reference level,” “control” and “reference” are used interchangeably and refer to the level of the relevant biomarker in healthy normal subject. For example, a “control” for determination as to whether or not a patient has an increased or decreased level of an HCN2 gene expression product is the level of the HCN2 gene expression product in a healthy normal subject.

A “healthy normal subject” is a subject that is asymptomatic of lower or upper urinary tract disorders.

“Sensory bladder disorder” means disorders of the urinary bladder where the primary dysfunction involves the sensory afferent nerves.

“Motor bladder disorder” means disorders of the urinary bladder where the primary dysfunction involves the motor efferent nerves, neuromuscular junction or muscle.

The terms “diagnosing” or “diagnosis of” or the like, include initial diagnosis and confirmation of a previous diagnosis.

A patient having symptoms of interstitial cystitis includes both patients that have not previously been diagnosed with interstitial cystitis and patients that have previously been diagnosed with and/or treated for interstitial cystitis.

A patient having symptoms of overactive bladder includes both patients having symptoms of overactive bladder that have not previously been diagnosed with overactive bladder and patients that have previously been diagnosed with and/or treated for overactive bladder.

A patient having symptoms of underactive bladder includes both patients having symptoms of underactive bladder that have not previously been diagnosed with underactive bladder and patients that have previously been diagnosed with and/or treated for underactive bladder.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

One embodiment of the present disclosure is a method for diagnosis of a disorder in a patient having one or more symptoms of interstitial cystitis comprising: (a) obtaining a biological sample from the patient, (b) measuring the level of an HCN2 gene expression product in the biological sample to obtain a biomarker measurement and (c) using the biomarker measurement to diagnose the disorder, wherein if the biomarker measurement indicates an increase in the level of the HCN2 gene expression product, the patient is diagnosed as having interstitial cystitis.

Another embodiment is a method for diagnosis of a disorder in a patient having one or more symptoms of overactive bladder comprising: (a) obtaining a biological sample from the patient, (b) measuring the level of an HCN2 gene expression product in the biological sample to obtain a biomarker measurement and (c) using the biomarker measurement to diagnose the disorder, wherein if the biomarker measurement indicates an increase in the level of HCN2 gene expression product, the patient is diagnosed as having overactive bladder.

Another embodiment is a method for diagnosis of a disorder in a patient having one or more symptoms of interstitial cystitis, overactive bladder, or underactive bladder comprising: (a) obtaining a biological sample from the patient, (b) measuring the level of an HCN2 gene expression product in the biological sample to obtain a biomarker measurement and (c) using the biomarker measurement to diagnose the disorder, wherein if the biomarker measurement indicates an increase in the level of HCN2 gene expression product, the patient is diagnosed as having interstitial cystitis, overactive bladder, or underactive bladder.

Another embodiment is a method for diagnosis of a disorder in a patient having one or more symptoms of interstitial cystitis, overactive bladder, or underactive bladder comprising: (a) obtaining a biological sample from the patient, (b) measuring the level of an HCN2 gene expression product in the biological sample to obtain a biomarker measurement and (c) using the biomarker measurement to diagnose the disorder, wherein if the biomarker measurement indicates an HCN2 gene expression product level higher than a control HCN2 level, the patient is diagnosed as having a sensory bladder disorder.

Another embodiment is a method for assessing the likely response to treatment in a patient having one or more symptoms of interstitial cystitis comprising: (a) obtaining a biological sample from the patient, (b) measuring the level of an HCN2 gene expression product in the biological sample to obtain a biomarker measurement and (c) using the biomarker measurement to assess whether the patient is likely or not likely to benefit from treatment.

Yet another embodiment is a method for assessing the likely response to treatment in a patient having one or more symptoms of overactive bladder comprising: (a) obtaining a biological sample from the patient, (b) measuring the level of an HCN2 gene expression product in the biological sample to obtain a biomarker measurement and (c) using the biomarker measurement to assess whether the patient is likely or not likely to benefit from treatment.

Overactive bladder (OAB) and interstitial cystitis present with common LUTS. Agrin, a multidomain heparan sulfate proteoglycan plays an important role in the development of the neuromuscular junction and acetylcholine receptor (AchR) clustering. Agrin is cleaved by neurotrypsin at 2 positions. Cleavage of agrin at the β-site releases C-terminal Agrin fragment (CAF, approx 22 kDa).

Still another embodiment is a method for diagnosing a patient having one or more symptom of interstitial cystitis, overactive bladder, or underactive bladder comprising (a) obtaining a biological sample from the patient, (b) obtaining a measurement of the level of c-terminal agrin fragment (CAF) in the biological sample to provide a CAF biomarker measurement, (c) using the CAF biomarker measurement to diagnose the patient as having interstitial cystitis, overactive bladder, or underactive bladder.

Yet another embodiment is a method for diagnosing a patient having one or more symptom of interstitial cystitis, overactive bladder, or underactive bladder comprising (a) obtaining a biological sample from the patient, (b) obtaining a measurement of the level of c-terminal agrin fragment (CAF) in the biological sample to provide a CAF biomarker measurement, (c) using the CAF biomarker measurement to diagnose the patient as having a motor bladder disorder if the CAF biomarker level is higher than a control level.

Another embodiment is a method for diagnosing a patient having one or more symptom of interstitial cystitis, overactive bladder, or underactive bladder comprising (a) obtaining a biological sample from the patient, (b) obtaining a measurement of the level of c-terminal agrin fragment (CAF) in the biological sample to provide a CAF biomarker measurement, (c) obtaining a measurement of the level of a HCN2 gene expression product in a biological sample, (d) comparing the level of a HCN2 gene expression product to an HCN2 control level to provide an HCN2 biomarker measurement, and (e) using both the CAF biomarker measurement and the HCN2 biomarker measurement to diagnose the patient as having interstitial cystitis, overactive bladder, or underactive bladder.

Yet another embodiment is a method for a selecting a treatment regimen for a patient having one or more symptoms of interstitial cystitis or overactive bladder comprising: (a) obtaining a biological sample from the patient, (b) measuring the level of an HCN2 gene expression product in the biological sample to obtain an HCN2 biomarker measurement and (c) using the HCN2 biomarker measurement to select a treatment regimen for the patient.

Another embodiment is a method for selecting a treatment regimen for a patient having one or more symptom of interstitial cystitis, overactive bladder, or underactive bladder comprising (a) obtaining a biological samples from the patient, (b) obtaining a measurement of the level of CAF in the biological sample to provide a CAF biomarker measurement, (c) using the CAF biomarker measurement to select a treatment regimen for the patient.

Still another embodiment is a method for selecting a treatment regimen for a patient having one or more symptom of interstitial cystitis, overactive bladder, or underactive bladder comprising (a) obtaining a biological samples from the patient, (b) obtaining a measurement of the level of CAF in the biological sample to provide a CAF biomarker measurement, (c) obtaining a measurement of the level of a HCN2 gene expression product in a biological sample, (d) comparing the level of a HCN2 gene expression product to an HCN2 control level to provide an HCN2 biomarker measurement, and (e) using both the CAF biomarker measurement and the HCN2 biomarker measurement to select a treatment regimen for the patient.

In some embodiments the patient's symptom is pelvic pain, or the patient has pressure and discomfort in the bladder and pelvic region, or the patient has frequent urination, or the patient has decreased force of urination, or the patient has increased urinary urgency, or the patient has urinary incontinence. A patient may have experienced one or more of the symptoms individually or simultaneously. In particular, in patients with interstitial cystitis the patient's symptoms include pain.

In certain embodiments the HCN2 gene expression product measured is HCN2 mRNA. In still other embodiments the HCN2 gene expression product measured is HCN2 protein.

The determination of whether the level of an HCN2 gene expression product is increased is determined by comparison of the HCN2 biomarker level to an HCN2 control level. The HCN2 control level is the level of HCN2 in a biological sample in a healthy normal subject. The levels of HCN2 biomarker level in different type of biological samples may vary. For example, the control levels in urine, tissue or serum. The HCN2 biomarker level may be based on a number of HCN2 gene expression products, such as the level of HCN2 protein or HCN2 mRNA. One of skill in the art can readily determine the level to use as the control level. For example, in a particular assay, such as an ELISA assay, urine samples from healthy normal subjects are analyzed for the level of HCN2 protein at the same time, or different times, than the patient samples. The number of biologic samples healthy normal subjects assayed is great enough to provide a control level with statistical significance. In general, the levels measured from healthy normal subjects will be within a range. Given the distribution of these levels a cut of level is determined and this number is used as the control number. Therefore, if the HCN2 biomarker measurement is determined by measuring the level of HCN2 protein and the biomarker measurement from a patient is higher than the control level, then the level of HCN2 protein the HCN2 protein is increased in the patient.

In certain embodiments the biological sample is a urine sample. In other embodiments the biological sample is tissue. In still other embodiments the biological sample is a tissue obtained from a biopsy of the bladder. In other embodiments the biological sample is a tissue obtained from a biopsy or the urethra of the patient.

The level of HCN2 protein may be measured using methods known in the art including the use of antibodies which specifically bind to an HCN2 protein or a fragment thereof. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art. For example, immunoassays, including immunohistochemistry, may be employed, e.g., an ELISA assay. Furthermore, mass spectrometry may be used to detect proteins or fragments thereof, and may be used in combination with other techniques such as HPLC.

The biological sample may also be analyzed for HCN2 mRNA using methods well known in the art including, but not limited to, PCR (polymerase chain reaction), RT-PCR (reverse transcriptase-polymerase chain reaction), quantitative PCR, etc.

In some embodiments the level of HCN2 mRNA in the biological sample is determined by real time PCR (qPCR). Method for designing primers for qPCR are well known in the art. For example, Beacon Designer™ (Premier Biosoft, Palo Alto, Calif.), Primer Quest (Integrated DNA Technologies, Coralvile, Iowa).

In particular embodiments the level of HCN2 mRNA in the biological sample is determined by qPCR wherein the forward and reverse primers used in the real-time PCR have the sequence 5′-CGCCTGATCCGCTACATCCT-3′ (SEQ ID NO:7) and 5′AGTGCGAAGGACTACAGTTCACT-3′ (SEQ ID NO:8) respectively.

Depending on the diagnosis of a patient, the patient is administered an agent that modulates the expression of an HCN2 gene expression product such as an HCN2 mRNA or HCN2 protein. In particular embodiments, wherein the patient is diagnosed with interstitial cystitis or overactive bladder, the patient is administered an HCN2 modulator that decreases expression of an HCN2 gene expression product. In one embodiment wherein the patient is diagnosed as having underactive bladder, the patient is administered an HCN2 modulator that increases the level of an HCN2 gene expression product. Therefore, another embodiment of the present disclosure is a pharmaceutical composition for treatment of interstitial cystitis and overactive bladder or underactive bladder comprising an effective amount of an HCN2 modulator.

One embodiment is a method of treating a patient with interstitial cystitis or overactive bladder comprising administering to the patient an HCN2 modulator which reduces the level of HCN2 mRNA. Another embodiment is a method of treating a patient with under active bladder comprising administering to the patient and HCN2 modulator which increase the level of HCN2 mRNA. One embodiment is a method of treating a patient with interstitial cystitis or overactive bladder comprising administering to the patient an HCN2 modulator which reduces the level of HCN2 protein. Another embodiment is a method of treating a patient with underactive bladder comprising administering to the patient and HCN2 modulator which increase the level of HCN2 protein.

One embodiment is a method for selecting a treatment regimen for a patient having one or more symptom of interstitial cystitis, overactive bladder, or underactive bladder comprising (a) obtaining a biological sample from the patient, (b) obtaining a measurement of the level of CAF in the biological sample to provide a CAF biomarker measurement, and (c) using the CAF biomarker measurement to select a treatment regimen for the patient.

Another embodiment is a method for selecting a treatment regimen for a patient having one or more symptom of interstitial cystitis, overactive bladder, or underactive bladder comprising (a) obtaining a biological sample from the patient, (b) obtaining a measurement of the level of CAF in the biological sample to provide a CAF biomarker measurement, (c) using the CAF biomarker measurement to select a treatment regimen for the patient, (d) obtaining a measurement of the level of a HCN2 gene expression product in the biological sample, (e) comparing the level of a HCN2 gene expression product to an HCN2 control level to provide an HCN2 biomarker measurement, (f) using both the CAF biomarker measurement and the HCN2 biomarker measurement to select a treatment regimen for the patient.

Another embodiment is a method for selecting a treatment regimen for a patient having one or more symptom of interstitial cystitis, overactive bladder, or underactive bladder comprising (a) obtaining a biological sample from the patient, (b) obtaining a measurement of the level of CAF in the biological sample to provide a CAF biomarker measurement, (c) using the CAF biomarker measurement to select a treatment regimen for the patient, (d) obtaining a second biological sample from the patient, (e) obtaining a measurement of the level of a HCN2 gene expression product in the second biological sample, (f) comparing the level of a HCN2 gene expression product to an HCN2 control level to provide an HCN2 biomarker measurement, and (g) using both the CAF biomarker measurement and the HCN2 biomarker measurement to select a treatment regimen for the patient.

The determination of whether the level of CAF is increased is determined by comparison to a CAF control level. In certain embodiments the CAF control level is the level of the CAF in a healthy normal subject. The levels of CAF in different type of biological samples may vary. For example the control level of CAF in urine is generally lower than the CAF level in serum. One of skill in the art can readily determine the level to use as the control level. For example, in a particular assay, such as an ELISA assay, urine samples from healthy normal subjects are analyzed at the same time, or different times, than the patient samples. The number of biologic samples healthy normal subjects assayed is great enough to provide a control level with statistical significance. In general, the levels measured from healthy normal subjects will be within a range. Given the distribution of these levels a cut of level is determined and this number is used as the control number. Therefore, if the level cut off level is 50 pM and the CAF biomarker measurement from a patient is higher than the control level, then the CAF level is increased in the patient.

An embodiment of the method for diagnosing a patient having one or more symptom of interstitial cystitis, overactive bladder, or underactive bladder, wherein the patient's HCN2 biomarker measurement is higher than control, the HCN2 modulator is an agent that lowers the level an HCN2 gene expression product. In some embodiments the HCN2 modulator is an antisense oligonucleotide that reduces the level of HCN2 gene expression or normalizes the HCN2 gene expression in the patient. In another embodiment In preferred embodiments the antisense oligonucleotide reduces the level of HCN2 protein. In particular embodiments the antisense oligonucleotide binds with high affinity to HCN2 mRNA in bladder and other bodily cavities accessible from outside of the patient's body. Methods for designing antisense molecules are well known in the art. For example, the antisense molecules of SEQ ID NOs:1-5 where designed using Mfold software and selected for their high score.

In some embodiments the antisense oligonucleotide has a length of 8, 9, 10, 11, 12, 13, 14, 15, 16, 17, 18, 19, 20, 21, 22, 23, 24, 25, 26, 27, 28, 29, or 30 nucleotides wherein the oligonucleotide is complementary to a human HCN2 mRNA sequence.

In some embodiments the oligonucleotide is selected from the group consisting of SEQ ID NOs:1-5, or functional fragments or variants thereof.

In some embodiments wherein the patient's HCN2 biomarker measurement is higher than control, the HCN2 modulator is an agent that lowers the level or activity of an HCN2 gene expression product. In one embodiment wherein the patient's HCN2 biomarker measurement is higher than an HCN2 control level, the HCN2 modulator is an agent that modulates the levels of intracellular cyclic AMP such as a phosphodiesterase inhibitor. In another embodiment wherein the patient's HCN2 biomarker measurement is higher than an HCN2 control level, the HCN2 modulator is a beta3 adrenoceptor agonist. In another embodiment wherein the patient's HCN2 biomarker measurement is higher than an HCN2 control level, the HCN2 modulator is an alpha 3 agonist. In other embodiments, wherein the patient's HCN2 biomarker measurement is higher than an HCN2 control level, the HCN2 modulator is a beta3 adrenorecteptor agonist selected from the group consisting of solabegron, rafabegron and mirabegron. In one embodiment wherein the patient's HCN2 biomarker measurement is higher than an HCN2 control level, the HCN2 modulator is an agent capable of directly binding an HCN2 channel (HCN2 gene expression product) or an agent capable of blocking an HCN2 channel activity. In another embodiment wherein the patient's HCN2 biomarker measurement is higher than an HCN2 control level the HCN2 modulator is an agent capable of directly binding an HCN2 channel protein selected from the group consisting of gabapentin or lamotrigine. In yet another embodiment wherein the patient's HCN2 biomarker measurement is higher than an HCN2 control level, the HCN2 modulator is an agent capable of blocking an HCN2 channel activity selected from the group consisting of zatebadine, clonidine, ivabradine and eugenol. In still another embodiment wherein the patient's HCN2 biomarker measurement is higher than an HCN2 control level, the HCN2 modulator is an antibody, or an antibody fragment, including but not limited to, an F(ab)₂, an Fab, a dimer of an Fab, an Fv, a dimer of an Fv, a scFv, a dimer of a scFv, a dimer an Fab, an Fv, a dimer of an Fv, a scFv, a dimer of a scFv, a trimer of an Fab, a trimer of an Fv, a trimer of a scFv, minibody, a diabody, a triabody, a tetrabody, a linear antibody, a protein, a peptide, an aptamer, a peptibody, an adnectin, or a nucleic acid, that binds to the HCN2 channel. In one embodiment wherein the patient's HCN biomarker measurement is lower than an HCN2 control level, the HCN2 modulator is an antibody or an antibody fragment that reduces the activity of the HCN2 channel protein. In another embodiment the HCN2 modulator is an antibody or antibody fragment that binds to the extracellular portion of the HCN2 channel. In still another embodiment wherein the patient's HCN2 biomarker measurement is lower that an HCN2 control level, the HCN2 modulator is an antibody or antibody fragment that increases the activity of the HCN2 channel protein.

In some embodiments the patient diagnosed with interstitial cystitis or overactive bladder is treated by administering an effective amount of an HCN2 modulator, for example, an antisense oligonucleotide that reduces the level of HCN2 gene expression or normalizes the HCN2 gene expression in the patient. In one embodiment wherein the patient's HCN2 biomarker measurement is higher than an HCN2 control level, the patient is administered an antisense oligonucleotide that reduces the level of HCN2 gene expression or normalizes the HCN2 gene expression in the patient. In another embodiment wherein the patient's HCN2 biomarker measurement is higher than an HCN2 control level, the patient is administered an antisense oligonucleotide that reduces the level of HCN2 gene expression or normalizes the HCN2 gene expression in the patient, wherein the antisense nucleotide is selected from the group consisting of SEQ ID NOs: 1-5, or functional fragments or variants thereof. Another embodiment of the present disclosure is a pharmaceutical composition for treatment of urinary symptoms associated with interstitial cystitis and overactive bladder comprising an effective amount of an antisense oligonucleotide that reduces the level of HCN2 gene expression or normalizes the level of HCN2 gene expression, and a pharmaceutically acceptable excipient, diluent, carrier, or adjuvant.

Embodiments of the present disclosure also include methods of treating or preventing symptoms associated with interstitial cystitis and overactive bladder in a patient in need thereof, comprising administering to the patient a pharmaceutical composition for treatment of symptoms associated with interstitial cystitis or overactive bladder comprising administering an effective amount of an antisense nucleotide selected from the group consisting of SEQ ID NOs: 1-5, or functional fragments or variants thereof, in combination with a pharmaceutically acceptable excipients.

Agrin, a synaptically located protein, is a key player during initial formation and maintenance of NMJs where it induces acetylcholine receptor (AChR) assembly and aggregation by binding to its receptor LRP4 followed by signaling through MuSK/DOK-7. During development, nerve cells approach muscle fibers and establish synaptic contacts defined as neuromuscular junctions (NMJ). Initially, the NMJ is small and weak, but if the contact is successful the connection is maintained and reinforced. Agrin induces and stabilizes clusters of AChRs at the NMJ, promotes synaptic maturation, and maintains the mature state of the NMJ.

Agrin is specifically cleaved by neurotrypsin, a serine protease, leading to a 22 kDa soluble C-terminal agrin fragment which is referred to in the literature as CAF or CAF22. CAF is present in blood and urine. Cleavage of agrin by neurotrypsin counteracts the synapse promoting activity of agrin. The molecular structure and the cleavage mechanism of serine proteases have been studied extensively and are well known. Both agrin and neurotrypsin are located in the synaptic cleft, the space between the pre-synaptic (signal-emitting) and the post-synaptic (signal-receiving) cell.

Agents useful in the treatment of patients that have levels of CAF higher than a control level, include agents that modify the levels or activity of agrin or increase or decrease the activity of neurotrypsin. For example in patient diagnosed as having interstitial cystitis or overactive bladder that have higher levels of CAF than control levels, may be treated by administering one or more agent that modulates the level of agrin or agrin activity. In particular embodiments, patients diagnosed as having interstitial cystitis or over active bladder are treated by administering one or more agent that decreases the level of agrin or agrin activity or an agent that increases the activity of neurotrypsin or blocks the interaction of agrin with LRP4. Patients diagnosed as having underactive bladder that do not have CAF levels higher than a control level, may be treated by administering agents that modulate the level of agrin or agrin activity. In particular embodiments, patients diagnosed as having underactive bladder that have CAF levels lower than control levels are treated by administering an agent that increases agrin, has agrin activity or that inhibits neurotrypsin.

Methods for determining whether a compound is a neurotrypsin inhibitor are known in the art, see for example, U.S. Pat. No. 7,897,364 and United States Patent Publication No. 2010/0240083, which are herein incorporated by reference.

Neurotrypsin inhibitors and methods for identifying inhibitors of neurotrypsin have been described, see for example, U.S. Pat. No. 7,897,363, United States Patent application publication US2013/0245064, and US2013/0261130, each of which are incorporated in their entirety herein. For example, US 2013/0261130 discloses neurotrypsin inhibitors including: 2-(3-Chlorobenzo[b]-thiophene-2-carboxamido)-4-(4-chloro-2-fluoro-phenyl-carbamoyl)benzoic acid, 2-(3-Chlorobenzo[b]-thiophene-2-carboxamido)-5-(4-chlorophenylcarbamoyl)-benzoic acid, 2-(4-(5-Chloro-1H-benzo[d]-imidazol-2-yl)benzamido)-5-(4-chlorophenylcarbamoyl)-benzoic acid, 5-(N-4-Butylphenyl-N-methyl-carbamoyl)-2-(4-(5,6-dichloro-1H-benzo[d]-imidazol-2-yl)benzamido)-benzoic acid, 5-(N-4-Chlorophenyl-N-isobutylcarbamoyl)-2-(4-(5,6-dichloro-1H-benzo[d]-imidazol-2-yl)benzamido)-benzoic acid, 5-(4-Chloro-2-fluorophenyl-carbamoyl)-2-(4-(5,6-dichloro-1H-benzo[d]-imidazol-2-yl)benzamido)-benzoic acid, 5-(4-Chlorophenyl-carbamoyl)-2-(4-(5,6-di-chloro-1H-benzo[d]imidazol-2-yl)benzamido)benzoic acid, 2-(4-(5,6-Dichloro-1H-benzo[d]imidazol-2-yl)-benzamido)-5-(diisobutyl-carbamoyl)benzoic acid, 5-(1-(4-Chlorophenyl)ethyl-carbamoyl)-2-(4-(5,6-dichloro-1H-benzo[d]-imidazol-2-yl)benzamido)-benzoic acid, and 5-(4-Chlorophenyl-carbamoyl)-2-(4-(5,6-dichloro-1-methyl-1H-benzo[d]imidazol-2-yl)-benzamido)benzoic acid. US US2013/0245064 discloses neurotrypsin inhibitors including N-(4-Chlorophenyl)-4-(4-fluorophenyl)-benzamido)-2-hydroxybenzamide, N-(4-Chlorophenyl)-5-(4-fluorophenyl)-thiophene-2-carbonyl)amino-2-hydroxybenzamide, N-(2,4-Difluorophenyl)-4-(4-(5,6-dichloro-1H-benzo[d]imidazol-2-yl)-benzamido)-2-hydroxybenzamide, N, N-Diisobutyl-2-hydroxy-4-((5,6-dichloro-1H-benzo[d]imidazol-2-yl)-benzamido)benzamide, and N-(4-Butylphenyl) 4-(4-(5,6-dichloro-1H-benzo[d]imidazol-2-yl)-benzamido)-2-hydroxybenzamide.

Agents having agrin activity have been described, see for example United States Patent Application Publication US2012/0208765 that is incorporated in its entirety herein.

In still another embodiment, the agent that modulates HCN2 function is an antibody, or an antibody fragment, including but not limited to, an F(ab′)₂, an Fab, a dimer of an Fab, an Fv, a dimer of an Fv, a scFv, a dimer of a scFv, a dimer an Fab, an Fv, a dimer of an Fv, a scFv, a dimer of a scFv, a trimer of an Fab, a trimer of an Fv, a trimer of a scFv, minibody, a diabody, a triabody, a tetrabody, a linear antibody, a protein, a peptide, an aptamer, a peptibody, an adnectin, or a nucleic acid, that binds to the HCN2 channel. In one embodiment the HCN2 modulator is an antibody or an antibody fragment that reduces the activity of the HCN2 channel protein. In another embodiment the HCN2 modulator is an antibody or antibody fragment that binds to the extracellular portion of the HCN2 channel. In still another embodiment HCN2 modulator is an antibody or antibody fragment that increases the activity of the HCN2 channel protein.

Drugs useful for treating patients having increased CAF levels include anticholinergic agents, e.g. Darifenacin (Enablex®) Fesoterodine (Toviaz®), Oxybutynin (Ditropan®, Ditropan XL®, Oxytrol®, Gelnique®), Solifenacin (Vesicare®), Tolerodine tartrate (Detrol®, Detrol Le), Trospium (Sanctura®). Therefore in one embodiment a patient having increased CAF levels is administered an anticholinergic agent. In certain embodiments, a patients having increased CAF level is administered an agent selected from the group consisting of Darifenacin Fesoterodine, Oxybutynin, Solifenacin, Tolerodine tartrate, and Trospium (Sanctura®).

Drugs useful for treating patients having increased CAF levels also include botulinum toxins, e.g., abobotulinumtoxinA (Dysport®), onabotulinumtoxinA (Botox®), incobotulinumtoxinA (Xeomin®), rimabotulinumtoxinB (Myobloc®). Therefore in one embodiment a patient having increased CAF levels is administered a botulinum toxin. In certain embodiments a patient having increased CAF level is administered a botulinum toxin selected from the group consisting of abobotulinumtoxinA, onabotulinumtoxinA, incobotulinumtoxinA and rimabotulinumtoxinB. In a preferred embodiment a patient having increased CAF levels is administered onabotulinumtoxinA.

Additional drug useful for treating patients having overactive bladder or interstitial cystitis include alpha-blockers, e.g tamsulosin (Flowmax™), doxazosin (Cardura™), terazosin (Hytrin™), alfuzosin (Uroxatral™). Therefore one embodiment wherein the patient has overactive bladder or interstitial cystitis the patient is administered an alpha-blockers. In one embodiment the alpha-blocker is selected from the group consisting of tamsulosin (Flowmax™), doxazosin (Cardura™), terazosin (Hytrin™), and alfuzosin (Uroxatral™).

Drugs useful in treating patients having elevated HCN2 levels also include neuroleptic drugs, e.g., gabapentin or lamotrigine; β3-adrenergic receptor agonists, e.g., solabegron or mirabegron (Myrbetriq®); nicotinic alpha 3 antagonists; Tofranil; nonsteroidal anti-inflammatory drugs, e.g., ibuprofen, naproxen, dexmecamylamine, or pentosan polysuifate (Elmiron®). Other treatments for patients having elevated HCN2 levels include bladder distention; bladder instillation of dimethyl sulfoxide (Rimso-50); bladder instillation of a solution comprising lidocaine, sodium bicarbonate and pentosan polysulfate; or bladder instillation of a solution comprising lidocaine, sodium bicarbonate and heparin.

### Formulations

In one aspect of the present invention, pharmaceutically acceptable compositions are provided, wherein these compositions comprise one or more HCN2 modulator or CAF modulator as described herein and a pharmaceutically acceptable carrier and, in addition, can include other pharmaceutical agents, adjuvants, or diluents. Also provided are pharmaceutically acceptable compositions that comprise one or more agents that modify the levels or activity of agrin or increase or decrease the activity of neurotrypsin. The composition may include one or more additional active ingredients such as an antimicrobial agent, anti-inflammatory agent, anesthetic and the like. In particular formulations, the HCN2 modulator or CAF modulator is a pharmaceutical composition for intravesical administration. In another formulation the compositions comprise a lipid or a liposome as a drug carrier. In preferred embodiments the composition is an orally targeted uroselective formulation without any cardiac or neuronal side effects.

Depending on the nature of the particular HCN2 modulator or CAF modulator, the modulator can be administered to humans and other animals, parenterally, (e.g., by intravenous or intraperitoneal injection), subcutaneously, orally, topically, rectally, buccally, as an oral or nasal spray. In particular embodiments the HCN2 modulator or CAF modulator is administered locally by instillation or injection in the patient's bladder. In other embodiments the HCN2 modulator or CAF is administered locally by instillation or injection into the patient's pelvic region.

The formulation may vary depending on the mode of administration. The pharmaceutical compositions can be in the form of solid, semi-solid, or liquid dosage forms, such as, for example, tablets, suppositories, pills, capsules, powders, liquids, suspensions, lotions, creams, gels, or the like, preferably in unit dosage form suitable for single administration of a precise dosage.

For the purposes of the present disclosure the term “excipient” and “carrier” are used interchangeably throughout the description of the present disclosure and said terms are defined herein as, “ingredients which are used in the practice of formulating a safe and effective pharmaceutical composition.” The formulator will understand that excipients are used primarily to serve in delivering a safe, stable and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients.

“Pharmaceutically acceptable” means a material that is not biologically or otherwise undesirable, i.e., the material may be administered to a patient without causing any undesirable biological effects or interacting in a deleterious manner with any of the other components of the pharmaceutical formulation in which it is contained. The carrier would naturally be selected to minimize any degradation of the active ingredient and to minimize any adverse side effects in the patient, as would be well known to one of skill in the art. See Remington's Pharmaceutical Sciences, 18th ed., Gennaro, Ark. Ed., Mack Publishing, Easton Pa. (1990), which discloses typical carriers and conventional methods of preparing pharmaceutical compositions that can be used in conjunction with the preparation of formulations of the agents described herein. It will be apparent to those persons skilled in the art that certain carriers can be more preferable depending upon, for instance, the route of administration and concentration of composition being administered.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate and the like. Liquid pharmaceutically administrable compositions can, for example, be prepared by dissolving, dispersing, etc., an active agent as described herein and optional pharmaceutical adjuvants in an excipient, such as, for example, water, saline aqueous dextrose, glycerol, ethanol and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art, for example see Remington's Pharmaceutical Sciences, 18th ed., Gennaro, Ark. Ed., Mack Publishing, Easton Pa. (1990).

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved, or dispersed in sterile water, or other sterile injectable medium prior to use.

The disclosed agents can also be present in liquids, emulsions, or suspensions for delivery of active therapeutic agents in aerosol form to cavities of the body such as the nose, throat, or bronchial passages. The ratio of agents to the other compounding agents in these preparations will vary as the dosage form requires.

### EXAMPLES

### Example 1

### HCN2 QPCR

Human bladders were obtained from organ donors with diagnosed interstitial cystitis and also from donors without urinary problems. Detrusor and urothelium were separated by microdissection under sterile conditions and stored in RNAlater® solution at −80° C. until RNA isolation. RNA was isolated from bladder tissues using RNeasy™ extraction kit according to the manufacturer protocol and quantified by a spectrophotometer. cDNA was synthesized by reverse transcription from 1 μg total RNA using RT2 PCR array first strand kit (SABiosciences™) and a highly sensitive quantitative Real Time Polymerase Chain Reaction (qRT-PCR) method for mRNA quantification was applied in a 25 μl mixture, which includes 12.5 μl RT2 Real-Time™ SYBR® Green/ROX PCR master mix (SABiosciences™), 10 μl nuclease-free H₂O, 10 μM specific human primers, including each of human HCN2 and human beta-actin as reference, and 1 μl template cDNA. Amplification was performed with an initial 10-minute step at 95° C., followed by 40 cycles at 95° C. for 15 seconds and at 60° C. for 1 minute using Mx3000P® qPCR detection system with fluorescence threshold values calculation by qPCR system software. Genomic DNA contamination in RNA samples was ruled out using controls without addition of Reverse Transcriptase during cDNA synthesis.

qPCR analysis of isolated cDNA from human bladder tissue revealed definite expression of HCN2 in urothelium and detrusor in both normal and IC bladders. Based on control values measured, the expression of HCN2 in IC bladder was higher in samples from the urothelium compared to normal bladder as shown in Table 1.

TABLE 1

| | |
|---|---|
| Bladder 23 (normal) urothelium | 24.15 |
| Bladder 24 (IC) urothelium | 25.1 |
| Bladder 25 (normal) urothelium | 22.31 |
| Bladder 23 (normal) detrusor | 24.05 |
| Bladder 24 (IC) detrusor | 22.59 |
| Bladder 25 (normal) detrusor | 21.81 |

### Example 2

### HCN2 Urine ELISA

A urine sample is collected from one or more patient and normal control subjects. The sample is immediately placed on ice after collection and centrifuges at 2400×gravity for 10 minutes. The supernatant is separated into 1.5 ml aliquots and preserved in a −80° C. freezer. One aliquot is used to measure the urinary creatinine (Cr) level. The urinary HCN2 gene expression product level is determine by an enzyme-linked immunosorbent assay (ELISA) method using an antibody sandwich format in 96-well plates. Each well of the plate is initially coated with a polyclonal anti-HCN2 antibody diluted in coating buffer and the plate is incubated overnight at 4° C. The next day, all wells are washed once with washing buffer and then blocking buffer is added to each well for 1 hour at room temperature to prevent any nonspecific binding. All wells are washed again and either a urine sample or an HCN2 standard is added to each well and incubated at room temperature for 6 hours with shaking. The experiment is done in triplicate. The wells are washed 5 times and monoclonal anti-HCN2 antibody diluted in blocking buffer is added to each well, and the plate is incubated overnight at 4° C. The following day, the wells are washed 5 times, and an anti-IgG antibody diluted in blocking buffer is added to each well and the plate is incubated for 2.5 hours with shaking at room temperature. The wells are washed 5 times and then incubated with substrate solution for 10 minutes at room temperature with shaking. Hydrochloric acid is added to terminate the reaction and the color change is measured at 450 nm using a Universal Microplate Reader. The amount of HCN2 gene expression product in each sample is calculated from the HCN2 standard curve. Each urinary HCN2 level sample measurement is normalized to its urine creatinine level, and is compared to the results among the different groups.

### Example 3

### C-Terminal Agrin Fragment (CAF) Urine ELISA Assay

Mid-stream urine specimens were collected from 7 female patients in the age range of 25-66 years and with clinical diagnosis of IC/PBS, 5 female and one male OAB patients in the age range of 25-87 years with symptoms of urinary frequency and urgency but without urinary incontinence. Urine was also collected from three asymptomatic female healthy volunteers in the age range 25-70 years.

CAF levels were measured using CAF Elisa Diagnostic Assay Kit from Neurotune (Product No. NT1001, Schlieren, Switzerland) and non-parametric statistical analysis was conducted. All steps were performed according to the manufacturer's directions except for the substitution of urine for serum samples. Briefly, calibration standards and urine samples were diluted and loaded into 96 well micro titer plates according to directions provided. The plates were incubated for 56° C. for 30 minutes followed by centrifugation for five minutes at 3000×g at room temperature. Ten μl of sample and calibration standards were transferred to the wells of Elisa micro titer plate that was precoated with an anti-CAF antibody. The transferred samples and calibration standards were mixed with 90 μl of dilution buffer previously added to the wells. The plates were incubated for 16 hours at room temperature. After washing the wells, a CAF Detector antibody solution was added and incubated for 30 minutes at room temperature. After washing the wells a detection agent was added and further incubated for 30 minutes. After washing and addition of a color development solution, the plate was read at 450 nm and data analyzed using the Excel analysis file provided by Neurotune.

We found that levels of CAF in the urine of controls were at or below the detection limit of the assay. In contrast, substantial elevation of CAF was noted in urine collected from patients with OAB or IC/PBS (Figure). Statistical analysis using Kruskal-Wallis followed by Dunn's test found over 65 fold significant elevation of CAF in urine of OAB patients relative to controls (326.2±164.3 pM vs 4.92±4.92 pM; p=0.028). Variability of CAF levels was noted in IC/PBS (243.7±139 pM), but it showed a trend towards significance.

These results show that agrin is expressed in the urinary bladder and we demonstrated that increased urine level of agrin C-terminal fragment in OAB and IC/PBS urine versus control. Agrin and the regulation of acetylcholine receptor regulation may play an important role in bladder contractility and offer a novel new insight into the pathophysiology in lower urinary tract symptoms (LUTS) that accompany.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the methods of this technology.

1. A method for diagnosing a patient having one or more symptom of interstitial cystitis, overactive bladder, or underactive bladder comprising the steps:
(a) obtaining one or more a biological samples from the patient;
(b) obtaining a measurement of the level of:
(i) a HCN2 gene expression product in the one or more biological samples to provide an HCN2 biomarker measurement; or
(ii) c-terminal agrin fragment (CAF) in the one or more biological samples to provide a CAF biomarker measurement; or
(iii) an HCN2 gene expression product in the one or more biological sample to provide an HCN2 biomarker measurement and a c-terminal agrin fragment (CAF) in the one or more biological samples to provide a CAF biomarker measurement; and
(c) using the HCN2 biomarker measurement and the CAF biomarker measurement, independently or in combination to diagnose the patient as having interstitial cystitis, overactive bladder, or underactive bladder.

2. A The method according to claim 1, wherein
the HCN2 biomarker measurement is used to diagnose the patient as having interstitial cystitis, overactive bladder, or underactive bladder.

3. The method according to claim 1, the method further comprising comparing the HCN biomarker measurement with an HCN2 control level and diagnosing the patient as having interstitial cystitis or overactive bladder if the HCN2 biomarker measurement is higher than the HCN2 control level.

4. The method according to claim 1, the method further comprising comparing the HCN biomarker measurement with an HCN2 control level and diagnosing the patient as having underactive bladder if the HCN2 biomarker measurement is less than the HCN2 control level.

5. The method according to claim 1, wherein
the CAF biomarker measurement is used to diagnose the patient as having interstitial cystitis, overactive bladder, or underactive bladder.

6. The method according to claim 5, the method further comprising comparing the CAF biomarker measurement with a CAF control level wherein if the CAF biomarker measurement is higher than the CAF control level, the patient is diagnosed as having a motor bladder disorder.

7. The method according to claim 1, wherein
both the CAF biomarker measurement and the HCN2 biomarker measurement are used to diagnose the patient as having interstitial cystitis, overactive bladder, or underactive bladder.

8. (canceled)

9. A method for selecting a treatment regimen for a patient having one or more symptom of interstitial cystitis, overactive bladder, or underactive bladder comprising the steps:
(a) obtaining one or more biological samples from the patient;
(b) obtaining a measurement of the level of:
(i) an HCN2 gene expression product in the one or more biological samples to provide an HCN2 biomarker measurement; or
(ii) c-terminal agrin fragment (CAF) in the one or more biological sample to provide a CAF biomarker measurement; or
(iii) an HCN2 gene expression product in the one or more biological samples to provide an HCN2 biomarker measurement and a c-terminal agrin fragment (CAF) in the one or more biological samples to provide a CAF biomarker measurement; and
(c) using the HCN2 biomarker measurement and the CAF biomarker measurement, independently or in combination to select a treatment regimen for the patient.

10. The method according to claim 9,
wherein the CAF biomarker measurement is used to select a treatment regimen for the patient.

11. The method according to claim 9,
(d)
wherein the HCN2 biomarker measurement is used to select a treatment regimen for the patient.

12. The method according to claim 9,
wherein both the CAF biomarker measurement and the HCN2 biomarker measurement are used to select a treatment regimen for the patient.

13. The method according to claim 9, the method further comprising
(d) comparing the level of the HCN2 biomarker measurement to an HCN2 control level;
wherein if the HCN2 biomarker measurement is higher than the HCN2 control level, the method further comprises administering to the patient an agent that decreases the level of HCN2 protein, an agent that decreases the activity of an HCN2 protein, an agent that reduces the level of intracellular cyclic AMP, a beta 3 adrenoreceptor agonist, pentosan polysulfate, a nonsteroidal anti-inflammatory drug, a neuroleptic drug, or any combination thereof.

14. The method according to claim 9, further comprising
(d) comparing the level of the HCN2 biomarker measurement with an HCN2 control level;
wherein if the HCN2 biomarker measurement is lower than the HCN2 control level the method further comprises: administering to the patient an agent that increases the level of HCN2 protein, an agent that increases the activity of an HCN2 protein, a phosphodiesterase inhibitor, or any combination thereof.

15. The method according to claim 10, further comprising
(d) comparing the level of the CAF biomarker measurement with a CAF control level wherein if the level of CAF biomarker measurement is higher than a CAF control level, the method further comprises: administering to the patient an agent that decreases the level of agrin, an agent that increases the activity of neurotrypsin, an anticholinergic drug, a botulinum toxin or any combination thereof.

16. The method according to claim 10, further comprising
(d) comparing the level of the CAF biomarker measurement with a CAF control level, wherein if the level of CAF biomarker measurement is not higher than a CAF control level the method does not comprise administering to the patient an agent that decreases the level of agrin, an agent that increases the activity of neurotrypsin, an anticholinergic drug, a botulinum toxin or any combination thereof.

17. The method according to claim 12, further comprising
(d) comparing the level of the HCN2 biomarker measurement with an HCN2 control level; and
(e) comparing the level of the CAF biomarker measurement with a CAF control level;
wherein if the level of CAF biomarker measurement is not higher than a CAF control level, and the level of HCN2 biomarker measurement is lower than an HCN2 control level, the method further comprises administering to the patient an agent that increases the level of agrin or inhibits the activity of neurotrypsin.

18. The method according to claim 13, the method further comprising
(e) comparing the level of the CAF biomarker measurement with a CAF control level;
wherein if the CAF biomarker measurement is higher than a CAF control level and the HCN2 biomarker measurement is higher than an HCN2 control level, the method further comprises administering to the patient an agent that decreases the level of agrin, an agent that increases the activity of neurotrypsin, an anticholinergic drug, a botulinum toxin or any combination thereof.

19. The method according to claim 13, the method further comprising
(e) comparing the level of the CAF biomarker measurement with a CAF control level;
wherein if the CAF biomarker measurement is not higher than a CAF control level and the HCN2 biomarker measurement is higher than an HCN2 control level, the method does not comprise administering to the patient an agent that decreases the level of agrin, an agent that increases the activity of neurotrypsin, a botulinum toxin or any combination thereof.

20. (canceled)

21. The method according to claim 18, wherein the agent is a botulinum toxin selected from the group consisting of abobotulinumtoxinA, onabotulinumtoxinA, incobotulinumtoxinA and rimabotulinumtoxinB; and the anticholinergic drug is selected from the group consisting of darifenacin, fesoterodine, oxybutynin, solifenacin, tolerodine tartrate, trospium and any combination thereof.

22. (canceled)

23. The method according to claim 13, wherein the agent that decreases the level of HCN2 protein is an antisense oligonucleotide and the antisense oligonucleotide has a sequence selected from the group consisting of ACTCCTCCAGCACCTCGTTG (SEQ ID NO:1), GCTTGCCAGGTCGTAGGTCA SEQ ID NO:2, ACTCCTCCAGCACCTCGTT (SEQ ID NO:3), CTTCATCTCCTTGTTGCCCT (SEQ ID NO: 4), GTACTCCTCCAGCACCTCGT (SEQ ID NO:5); a functional fragment of SEQ ID NO:1, a functional fragment of SEQ ID NO:2, a functional fragment of SEQ ID NO:3, a functional fragment of SEQ ID NO:4 and a functional fragment of SEQ ID NO:5.

24-32. (canceled)
